(19) United States
(12) Patent Application Publication
(10) Pub. No.: US 2019/0388353 A1
(43) Pub. Date: Dec. 26, 2019

(54) ORGANIC TABLET FORMULATION

(71) Applicants: Max Spielberg, Beverly Hills, CA (US); David Johnson, Beverly Hills, CA (US)

(72) Inventors: Max Spielberg, Beverly Hills, CA (US); David Johnson, Beverly Hills, CA (US)

(21) Appl. No.: 16/014,171

(22) Filed: Jun. 21, 2018

(51) Int. Cl. A61K 9/20; A61K 9/00

(52) U.S. Cl. CPC A61K 9/2068; A61K 9/0056; A61K 9/2054; A61K 9/2059; A61K 9/2095; A61K 9/2018

(57) ABSTRACT

A chewable tablet includes:
an active ingredient combined with organic dextrose, organic Nu Rice Bran, organic Acai Berry, organic carnauba wax, organic maltodextrin.

### BACKGROUND OF THE INVENTION

For many years, chewable tablets have been a preferred and highly sought after dosage form in both pharmaceutical and nutritional supplements.

Although there are many chewable tablet products available, there is a market segment that has been left unaddressed.

Until this invention, there were no certified organic chewable pharmaceutical tablets available. One reason for this includes the fact that commonly known and used binders and lubricants are synthetic.

The present invention has discovered novel formulations for addressing this current need.

### SUMMARY OF THE INVENTION

In one embodiment, the present invention is a chewable tablet formulation comprising:

an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:6-13.

In one embodiment, the active ingredient is an active pharmaceutical ingredient, a nutritional supplement, or combinations thereof.

In one embodiment, the active ingredient is an active pharmaceutical ingredient.

In one embodiment, the organic maltodextrin:active ratio is 1:8—The present invention also includes a method of forming a chewable tablet as disclosed herein, said method comprising the steps of:

preparing a trituration component of organic maltodextrin:active ratio in a ratio of about 1:6-13;

combining said trituration component into a mixture with organic dextrose, organic Nu Rice Bran, organic Acai Berry, organic carnauba wax;

compressing said combined mixture into a chewable tablet.

In one embodiment, the method includes preparing a trituration component of the ratio of organic maltodextrin:active ratio is 1:8-11.

In one embodiment, the present invention is a chewable tablet formulation consisting of:

an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:6-13.

In one embodiment, the present invention is a chewable tablet formulation consisting of:

an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:8-11.

In one embodiment, the present invention is a tablet formulation comprising:

an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:6-13.

In one embodiment, the active ingredient in the tablet is an active pharmaceutical ingredient, a nutritional supplement, or combinations thereof.

In one embodiment, the active ingredient in the tablet is an active pharmaceutical ingredient.

In one embodiment, the organic maltodextrin:active ratio is 1:8—The present invention also includes a method of forming a chewable tablet as disclosed herein, said method comprising the steps of:

preparing a trituration component of organic maltodextrin:active ratio in a ratio of about 1:6-13;

combining said trituration component into a mixture with organic dextrose, organic Nu Rice Bran, organic Acai Berry, organic carnauba wax;

compressing said combined mixture into a chewable tablet.

In one embodiment, the method includes preparing a trituration component for a tablet of the ratio of organic maltodextrin:active ratio is 1:8-11.

In one embodiment, the present invention is a tablet formulation consisting of:

an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:6-13.

In one embodiment, the present invention is a tablet formulation consisting of:

an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:8-11.

### DETAILED DESCRIPTION OF THE PREFERRED EMBODIMENT

Persons with knowledge of solid dosage chewable tablet formulations are familiar with the various excipients available to the formulator. These include those materials that are listed in the US Food and Drug Administration guide—Generally regarded as safe (GRAS). The most common binders are polymers such as polyvinylpyrrolidone (PVP) and polyethylene glycol (PEG).

Although much of the formulation development relates to chewable tablets, if has also been discovered that non-chewable tablets are also functional under the various aspects of the invention as disclosed herein.

It is known and understood, that one who is trying to make an organic product cannot use synthetic polymers.

The present invention has discovered that carnauba wax and tapioca dextrose in combination, as the only binding agents in a tablet will effectively bind materials compressed into a chewable tablet.

An additional formulation difficulty relates to the use of lubricants. Most commonly, materials utilized include: talc, silica, magnesium stearate or stearic acid. All of the commonly used tablet lubrication excipients are artificial and cannot be used in an organic chewable tablet.

The present invention has discovered that carnauba wax will function as both a lubricant for our chewable tablets, as well as for a tablet compression agent. Furthermore, although there are some compression systems utilizing a particular excipient to impart multiple functions in a tablet formulation, the present invention has discovered an additional unknown formulation.

The present invention has further discovered formulation of active ingredients in maltodextrin through trituration. This is the first time anyone has inserted active drugs in maltodextrin to use the maltodextrin as an agent to transfer the actives into the tablet. When actives are placed in the maltodextrin, the maltodextrin is heated to 180 degrees to assist with settling of the actives. The actives are then blended in the chewable tablet.

The present invention has discovered that there is a criticality in utilizing particular ratios that have been demonstrated to impart the desired properties. It is not merely percentage composition, as chemistry still remains an art where there are many observed system phenomena that are not fully understood. Thus, the ratio disclosed herein is not merely the conclusion of simple experimentation.

In one embodiment, the maltodextrin:active ratio is prepared in a ratio of about 1:6-13. In a preferred embodiment, the ratio is about 1:8-11. The trituration includes drying the maltodextrin active combination at negative pressure for 30-60 min at 200-300 mbarr.

The present invention is exemplified in the following examples:

### Example 1

Organic Dextrose, Croscarmellose Sodium, Microcrystalline Cellulose, Lactose, Organic Acai Berry Flavor

Tablets compressed but did not exhibit satisfactory friability.

### Example 2

Organic Dextrose, Microcrystalline Cellulose, Lactose, Organic Acai Berry Flavor

Besides the Organic Sugar (Dextrose), there is not much choice for lubrication (Silicon Dioxide, Magnesium Stearate) due to the need for the product to be 100% certified organic/natural ingredients, and therefore there are sticking problems of the tablets to the punches and the edges of the tablets are also not even.

Tablets exhibit variable compression characteristics.

### Example 3

Organic Dextrose, Microcrystalline Cellulose, Lactose, Organic Nu Rice Bran, Carnauba Wax, Organic Acai Berry

Tablets compressed but did not exhibit satisfactory friability.

### Example 4

Organic Dextrose, Microcrystalline Cellulose, Organic Nu Rice Bran, Organic Carnauba Wax, Organic Acai Berry, Organic Rice Hull

Tablets exhibited satisfactory compression and friability.

Organic Rice Hull was added but it affected the taste & color. Adding the Organic Carnauba Wax did not have much of an effect.

### Example 5

Organic Dextrose, Microcrystalline Cellulose, Organic Nu Rice Bran, Organic Carnauba Wax, Organic Acai Berry, Organic Maltodextrin

Organic maltodextrin used instead of the rice hull we have been using. Tablets exhibited satisfactory compression and friability.

However, microcrystalline cellulose is undesired as it's a synthetic ingredient.

### Example 6

Organic Dextrose, Organic Nu Rice Bran, Organic Acai Berry, Organic Carnauba Wax, Organic Maltodextrin.

Tablets exhibited satisfactory compression and friability.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A chewable tablet formulation comprising:
an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:6-13.

2. The tablet formulation of claim 1, wherein the active ingredient is an active pharmaceutical ingredient, a nutritional supplement, or combinations thereof.

3. The tablet formulation of claim 1, wherein the active ingredient is an active pharmaceutical ingredient.

4. The tablet formulation of claim 1, wherein the organic maltodextrin:active ratio is 1:8-11.

5. A method of forming a chewable tablet of claim 1, said method comprising the steps of:
preparing a trituration component of organic maltodextrin:active ratio in a ratio of about 1:6-13;
combining said trituration component into a mixture with organic dextrose, organic Nu Rice Bran, organic Acai Berry, organic carnauba wax;
compressing said combined mixture into a chewable tablet.

6. The method of claim 5, wherein the ratio of organic maltodextrin:active ratio is 1:8-11.

7. A tablet formulation comprising:
an active ingredient, organic dextrose, organic Nu Rice Bran, organic Acai Berry, organic carnauba wax, organic maltodextrin wherein the organic maltodextrin:active ratio is prepared by trituration prior to tableting in a ratio of about 1:6-13.

8. The tablet formulation of claim 7 wherein said active ingredient in the tablet is an active pharmaceutical ingredient, a nutritional supplement, or combinations thereof.

9. The tablet formulation of claim 7 wherein said active pharmaceutical ingredient.

10. The tablet formulation of claim 7 wherein said organic maltodextrin:active ratio is 1:8-11.
